(19) United States
(12) Patent Application Publication
(10) Pub. No.: US 2022/0142897 A1
(43) Pub. Date: May 12, 2022

(54) Formulation and Use Thereof in the Treatment of Keratosis Pilaris

(71) Applicant: Devin Beaubien, Plano, TX (US)

(72) Inventor: Devin Beaubien, Plano, TX (US)

(21) Appl. No.: 17/525,252

(22) Filed: Nov. 12, 2021

Related U.S. Application Data
(60) Provisional application No. 63/112,229, filed on Nov. 11, 2020.

(51) Int. Cl. A61K 8/63; A61K 8/68; A61K 8/36; A61K 8/92; A61Q 19/00

(52) U.S. Cl. CPC A61K 8/63; A61K 8/68; A61Q 19/007; A61K 8/922; A61K 8/361

(57) ABSTRACT

The present invention generally relates to a formulation for the treatment of various skin conditions, including but not limited to keratosis pilaris wherein the main ingredient of the formulation comprises of DHEA. Embodiments of the invention can comprise of 0.1%-100% DHEA. Alternate embodiments include formulation, and treatment thereof, further comprising of lipids, niacinamides, ceramides, and/or cholesterol while the inactive ingredients may comprise of distilled water, emulsifying wax, oil, vitamin E, and glycerin.

### FIELD OF THE INVENTION

The present invention relates to a compound and formulation for treatment of dermatological conditions in accordance with recommended dosage. Specifically, the compound comprises of a topical cream comprised of dehydroepiandrosterone (DHEA). The present invention can be utilized for a variety of skin conditions, including keratosis pilaris (KP).

### RELATED APPLICATION

This application claims the benefit of U.S. Provisional Patent Application No. 63/112,229 filed Nov. 11, 2020, which is hereby incorporated by reference in its entirety.

### SUMMARY OF THE INVENTION

According to embodiments of the current invention, a topical cream treatment for skin conditions comprising of DHEA. In embodiments, the formulation may comprise of 0.1% to 10% DHEA by weight. In the preferred embodiment, the formulation comprises of 2% DHEA by weight. According to certain embodiments, the topical cream further comprises of at least one additional element from a group comprising of niacinamide, ceramides 1&3, free fatty acids, and cholesterol.

According to a select embodiment of the current invention, a topical cream comprising of 2% DHEA, 5% niacinamide, 1% lipids, 1% cholesterol, and 2% ceramides by weight where the remaining amount is comprised of distilled water.

According to other embodiments, the topical cream comprises of DHEA and a ceramide cholesterol complex, wherein said complex comprises of ceramides 1 & 3, cholesterol, and a free fatty acid. According to certain embodiments, the topical cream comprises of DHEA and ceramide cholesterol complex which itself comprises of about 1% ceramides 1 & 3; 2% cholesterol and 1% sunflower seed oil by weight. In other embodiment, the ceramide cholesterol complex comprises of 1% ceramides 1 & 3; 1% cholesterol and 1% sunflower seed oil by weight. In other embodiment, the ceramide cholesterol complex comprises of 5% ceramides 1 & 3; 1% cholesterol and 2% sunflower seed oil by weight.

According to an alternative embodiment of the current invention, a topical cream comprising of DHEA, distilled water, niacinamide, vegetable glycerin, oil, ceramide cholesterol complex, and a preservative. The ceramide cholesterol complex may comprise of at least 3% weight. In other embodiments, the topical cream may further comprise of emulsifier wax. In select embodiments, the oil used may be jojoba oil and/or cranberry seek oil. In certain embodiments the preservative used is AMTicide coconut.

According to embodiments of the invention, the topical cream may comprise of additional skin treatment formulations such as alpha-hydroxy acids (AHAs).

According to embodiments, the topical cream may be used as a part of a treatment regiment for KP. The method of treating KP comprises of applying a topical cream between two to three times daily wherein said topical cream comprises of DHEA. Some embodiments include application of the cream after showering. Other embodiments require exfoliation of the skin before application of the topical cream. Exfoliation can be physical (e.g. using a topical scrub comprising of grainy particles), mechanical (e.g. using exfoliation tool), or chemical (e.g. alpha and beta-hydroxy acids).

### BACKGROUND OF THE INVENTION

Keratosis Pilaris is a skin condition characterized by the appearance of small, rough follicular papules (or bumps) on the surface of the skin. On occasion the papules can be itchy, particular during the colder months when there is comparatively less atmospheric moisture. In addition to the papules, KP is accompanied with skin discoloration in the form of pink, purple, red, brown, or black dots.

The papules caused by KP are believed to be the result of excess formation of the skin protein keratin. Keratin is a key structural protein in the formation of hair and nails. The papules are generated when excess keratin surrounds hair follicles leading to the formation of hair plugs.

The pathogenesis of KP is currently unknown. While KP may have genetic components, no genes have been identified that are believed to be responsible for this condition. Nonetheless, it is believed that aberrant activity at the cellular level of KP patients manifests into physical conditions. Specifically, defects in the epithelial barrier, reduction in lamellar body secretions, and atrophy of the sebaceous glands correlate with the appearance of lesions/papules, follicular erythema, general surface erythema, skin sensitivity, capped hair follicles, and abnormal trans epidermal water loss.

In healthy, normal skin cells, keratinocytes, which are skin cells in the outermost epidermis layer, secrete lamellar bodies which form an impermeable, lipid-containing membrane that forms a skin barrier. Lamellar bodies also release components such as various lipids (e.g. glucosylceramides), hydrolytic enzymes (e.g. proteases, phosphatases, glucosidases, lipases), and proteins (e.g. corneodesmosin) that facilitate skin shedding (desquamation) of the epidermal layer. However, in individuals with KP, the lamellar bodies mis-secrete their contents. As a result skin cells fail to scaffold and build a broken epithelial barrier. Because the outermost epidermal surface is not properly formed, basal cells in the skin layer below attempt to correct the mis-developed skin by producing more keratinocytes to procure more lamellar bodies at the expense of producing sebocytes. Since the affected skin is busy with producing keratinocytes, the sebaceous glands atrophy, leading to dry skin. Meanwhile the excess keratinocytes lead to excess keratin, which eventually create the papule or lesions observed as inflamed tactile protrusions (or bumps) on the skin.

To date, treatment of KP is limited to treating the external symptoms on skin surface. Thus, it is an objection of the current invention to not just treat the external symptoms of KP but the condition itself at the cellular and microscopic level. The current invention is specifically formulated to correct the molecular and cellular defects in the skin to yield more effective treatment. It is also an object of the current invention to provide for a safe yet effective means for the treatment of KP. This and other features and advantages of the present invention will be explained and will become apparent to one skilled in the art through the summary of the invention that follows.

### BRIEF DESCRIPTION OF THE DRAWING

FIG. 1 is a bar graph quantifying the number of participants who observed certain effects based on visual detection by the human eye.

### DETAILED SPECIFICATION

The present invention generally relates to a formulation for the treatment of skin conditions. In the preferred embodiment, the formulation comprises of approximately 2.0% DHEA. In alternate embodiments, the current invention comprises of a formulation that contains approximately 0.1%-10% DHEA. In further alternate embodiments, the formulation comprises of DHEA and at least one of the following: niacinamide, ceramides 1& 3, cholesterol, and additional lipids.

The main component of the current invention is DHEA, a steroid hormone. Without being bound by theory, it is hypothesized that DHEA in the current invention serves to simulate the production of sebaceous glands. At 2% DHEA, there is an increase in sebum production as observed by increase in softness and oiliness. Such phenotypes are observed even when the percentage of DHEA is reduced to approximately 0.1%. However, clinical studies show that concentrations of DHEA at less than 0.1% are ineffective at stimulation of the sebaceous glands. In contrast, the upper limits of DHEA in the current invention can be as high as 100%.

Alternate embodiments of the current invention include at least one of the following: niacinamide, ceramides, cholesterol, and lipids. In the preferred embodiment the formulation comprises of each of the aforementioned components as follows: approximately 5% of the formulation constitutes niacinamide, approximately 1.0% of the formulation constitutes ceramides 1 & 3, approximately 1.0% of the formulation constitutes lipids, and approximately 2.0% of the formulation constitutes cholesterol. Alternate embodiments include a formulation without niacinamide. Specifically, in an embodiment without niacinamide, the formulation comprises of at least 0.1% DHEA, 1% lipids, 2% cholesterol, and 1% ceramide. However, alternate embodiments, may include approximately 0.5%-3% ceramides (1 & 3), 0.5%-3% lipids, and 1%-5% cholesterol. Without being bound by theory, it is believed that these additional compounds mirror the lamellar body secretions that are absent in patients with KP. These secretions are necessary for healthy skin since they create tight junctions and structures which create a barrier on the skin and decrease moisture loss. In patients with KP, the lack of such barrier creates an opportunity for allergens and irritants to pass through causing an immune response and inflammation.

According to embodiments of the current invention, references to “lipids” in the formulation of the current embodiment may include free fatty acids. In certain embodiments, the lipids can be oil. As example, linoleic oils such as rose hip oil, grape seed oil, cranberry oil, buckthorn oil, and sunflower seed oil are suitable for use in the current formulation. Other lipids that can be used in embodiments of the formulation include squalene, which is derived from shark liver oil. Embodiments of the invention may include a single type of lipid or a combination of lipids.

In certain alternate embodiments of the current invention, the formulation comprises of approximately 5% niacinamide. In further alternate embodiments, the formulation comprises of 0.1%-7% niacinamide. It is believed that niacinamide facilitates a reduction in inflammation and hyperpigmentation while promoting natural ceramide production within skin cells. According to clinical trials, it is believed that niacinamide improves the skin texture and appearance of KP patients by reducing hyperpigmented spots.

According to embodiments of the current invention, the formulation is a topical cream. In the preferred embodiment, the topical cream contains components that facilitate the delivery of the formulation through the skin. As examples, the topical cream can comprise of one or more of the following elements: distilled water, emulsifying wax, oil, vitamin E, and glycerin.

Embodiments of the current invention may employ base agents such as creams, lotions, oils, moisturizers, gels or ointments. Examples of inactive base agents or components include, for example, phospholipon 90H, lanolin, t5 hydrophilic ointment, white ointment, yellow ointment, polyethylene glycol ointment, petrolatum, hydrophilic petrolatum, white petrolatum, rose water ointment, squalene, hydrogenated vegetable oil (Type I), ultrasound gel, pluronic lecithin organogel (PLO) gel, cream. The term “petrolatum” as used herein means petrolatum ointment, petrolatum gel or 30 petrolatum cream, all of which are commercially available. It is well within the realm of the ordinary practitioner of pharmaceutical arts to determine which form of petrolatum is most appropriate for a specific kit.

Further embodiments of the current formulation comprise of active ingredients that provide other beneficial effects to the skin. As one example, improvements in the current formulation include combining a formulation embodiment with alpha-hydroxy acids (AHAs). AHAs are believed to smooth fine lines, reduce surface wrinkles, improve skin texture and tone, unblock clogged pores, and improve overall skin condition and tone. As a second example, an improvement in the current formulation includes combining a formulation with lactic acid. The addition of lactic acid may aid in maintaining skin moisture and reducing sun spots and age spot.

There are numerous quantifiable phenotypes that indicate the degree of severity of KP. Among the quantifiable phenotypes that may distinguish mild cases of KP from severe cases of KP include the number of lesions within a specific area (e.g. 4 cm×4 cm square); the extent to which KP affects the extremities and trunk; the amount of lesions/papules that rise vertically off of the skin; and the surface area of the skin affected by erythema and inflammation.

According to embodiments of the current invention, methods of treating KP wherein a formulation comprising of DHEA is administered to a patient in amounts sufficient to clear the skin are provided. Clearing of the skin is defined as visible and tactile reduction of at least one of the following: papules/lesions, follicular erythema, inflammation, and overall tones of redness in the skin. According to testing, a formulation comprising of 2% DHEA was able to yield 90%-95% clearness of skin by eleven (11) days. The patient was administered 2-3 topical applications on the KP affected skin. The patient also followed a regimen of showering once a day, either in the morning or evening to exfoliate and pretreat the skin. The patient would use a loofa or a body scrub to exfoliate. Immediately after showering, the patient applied the formula. The patient would typically apply the formulation to their skin once in the morning and once at night, or every 12 hours.

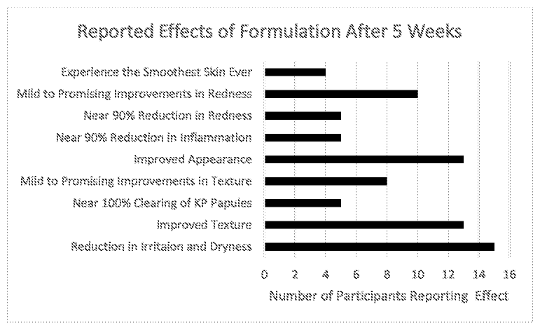

According to embodiments of the current invention, methods of treating KP wherein a formulation comprising of DHEA is administered to a patient in amounts sufficient to make the skin softer. As FIG. 1 illustrates, approximately 27% of participants in a trial test for the formulation reported that their skin has never been smoother after treatment for 5 weeks. Such trial test was performed using a formulation comprising of 10% DHEA. Patients were asked to pretreat their skin by exfoliating in the shower. Following pretreatment, patients applied approximately 21 millimeter (mm) in diameter and 2 mm in thickness (collectively, “nickel-size”) of a 2% DHEA formulation. The formulation was applied to patients at twice a day, at approximately 12-hour intervals.

According to embodiments of the current invention, methods of treating KP wherein the formulation comprising of DHEA is administered to a patient in amount sufficient to reduce the number of lesions within a specific area. In a preferred embodiment, a formulation of the current invention comprising of 2%-10% DHEA is applied to the infected area twice a day. A fifty percent (50%) reduction is expected using formulation 2 as presented below within nine (9) days.

According to embodiments of the current invention, methods of treating KP wherein the formulation comprising of DHEA is administered to a patient in an amount sufficient to reduce the size of the lesions/papules. In an exemplary embodiment using formulation 2 as presented below, users were able to notice significant decrease in size between day 1 and day 9 when the formulation was applied twice a day.

According to embodiments of the current invention, methods of treating KP wherein a formulation comprising of DHEA is administered to a patient in amounts sufficient to restore atrophied sebaceous glands. From a cosmetic perspective, atrophied sebaceous glands result in dry and rough skin. Accordingly, an indirect means of measuring atrophied sebaceous glands is to treat the skin so it becomes smooth and pliable. In an exemplary embodiment, a method of restoring atrophied sebaceous glands comprises of treating an area afflicted with KP with formulation 2 for 5 weeks. The formulation is applied twice per day and each application follows exfoliation of the skin. The treatment period may be extended if positive results are observed but the desired effect has not been achieved.

According to embodiments of the current invention, the formulations and methods can be applied to any area where the patient experiences symptoms of KP. Such areas are typically on the arm, thighs, hips, rib cage, and buttocks.

According to embodiments of the current invention, the method comprises of treatment of KP wherein the formulation comprising of DHEA is applied at least once a day. In alternate embodiments, the formulation is applied twice a day. In as yet further embodiments, the formulation is applied up to three times per day. In the preferred embodiment, methods of treatment entail application of the formulation once in the morning and once before going to bed.

According to the preferred embodiment of the current invention, a method comprises of treatment of KP wherein the formulation comprising of DHEA is applied at least once a day for a span of one week. In alternate embodiments, the treatment is applied for a span of up to six months. The duration of treatment is not a limiting feature of the current invention since the time frame can vary depending on the severity of KP.

According to embodiments of the current invention, a formulation comprising of DHEA is administered to patients after showering or bathing.

According to embodiments of the current invention, enhancements to the method include application of the formulation after exfoliation. Such action removes excess keratin and improves the rate of absorption of the formulation.

According to embodiments of the current formulation and method, treatment is applied to animals, including humans. The treatment is suitable for both males and females, children and adults.

According to embodiments of the current invention, a method of stimulating sebocytes and sebaceous glands is comprised of treating an affected area on the skin with an effective amount of DHEA.

According to embodiments of the current invention, a method of treating KP using DHEA is pre-administered, co-administered, or post-administered with other treatments of KP.

Turning now to the illustrations, FIG. 1 is a graphical representation of the effects of a trial testing a formulation. According to the tested embodiment, formulation 2 as described below was tested. The results presented in FIG. 1 are a summary of anecdotal observations made by the human eye. The trial consisted of fifteen (15) subjects, with eight (8) female subjects and seven (7) male subjects. The subjects comprised of persons of diverse racial backgrounds. The test trial spanned five (5) weeks. Each subject was considered to have moderate to severe lesional KP based on the area of lesions, number of lesions per area, and redness and inflammation of skin. Subjects followed a regimen of exfoliation in the shower, followed by application of formulation 2 in an amount sufficiently cover the treatment area by 0.5 mm-3 mm twice a day, morning and night. The results based on anecdotal reports of the subjects are presented. The x-axis represents the number of subjects who observed the phenotypes listed along the y-axis. “Mild improvements” is defined as improvements in texture, lessening of raised bumps, and slower formation of additional bumps/lesions. “Promising improvements” is defined as improved skin texture that is resilient to exfoliation and re-growth of hair in areas previously occupied by keratin blockages in the follicular channel.

In brief, FIG. 1 illustrates that all fifteen (15) subjects experienced a reduction in skin irritation and dryness, and thirteen (13) subjects reported improved skin texture and appearance. Five (5) subjects reported drastically improved texture and appearance, namely a reduction in KP papules clearing from the skin and over 90% reduction in inflammation and redness. Within the group that reported drastic improvements, four subjects stated that their skin has been the smoothest it has ever been. All subjects reported improvement in texture before detecting any reduction in redness. Of the two subjects that did not observe any improvements in texture and appearance, one subject (a male in his 20s) had previously undergone treatment with isotretinoin but developed KP as a result of isotreinoin. The other subject who did not observe any improvements in texture and appearance had other co-existing skin conditions in addition to KP.

Based on the aforementioned trial, it has been observed that improvements can appear at different rates of change. Some participants observed results within two to three weeks while the majority of subjects did not observe any effects until week 4 or week 5. Without being bound by theory, it is believed that factors such as sleep patterns, daily hydration, and exfoliation regimen can affect the intensity of effects and how early the effects may be visible. However, one subject reported different rates of effects different parts of her body; the subject observed results on her face at week 2 but observed results on her arms later.

DHEA has been shown to be safe for continuous topical application at low concentrations to avoid systemic circulation. Daily applications of DHEA can increase the output of sebaceous glands by up to 60% within 3 months. This makes DHEA a particularly viable and effective treatment for increasing sebum output in those with KP. Patients with KP typically have widespread sebaceous gland atrophy in the affected areas of the skin, causing downstream symptoms of skin dryness, inflammation, and could potentially be the root cause of the keratin papules that develop. DHEA also is suspected to help modulate and regulate keratinocyte proliferation. It is suspected that by regulating keratinocytes in the follicular channel, the rate at which keratin blockages forms is reduced.

Without being bound by theory, it is believed that the current formulation when applied to the skin may reverse sebaceous gland atrophy and supplement the skin's lamellar body lipid secretion. DHEA is believed to up-regulate the conversation of the hormone testosterone to DHT (dihydrotestosterone) through 5a-reductase, an enzyme involved in steroid metabolism. DHEA may itself be reduced to DHT by 5a-reductase. Sebaceous glands are particularly sensitive to DHT and increased levels of DHT promote the production of sebum. Without wishing to be bound by theory, it is believe that the increase in sebum production by DHEA eliminates an underlying cause for the dry, patchy skin of a KP patient.

### EXEMPLARY EMBODIMENTS

Formulation 1: According to a first formulation, the formulation comprises of 0.1% DHEA while the remaining ingredient is the inactive phospholipon 90H.

Formulation 2: According to a second formulation, the formulation comprises of 2% DHEA while the remaining ingredient is in the inactive phospholipon 90H.

Formulation 3: According to a third formulation, the formulation comprises of 2% DHEA, 1% cholesterol, 2% ceramides, and 1% rosehip oil while the remaining ingredient is the inactive phospholipon 90H.

Formulation 4: According to a fourth formulation, the formulation comprises of 2% DHEA, 2% cholesterol, 1% ceramides 1 and 3, 1% safflower oil, and 5% niacinamide while the remaining ingredient is the inactive phospholipon 90H.

Test of Formulation 1—According to a first exemplary embodiment, a male patient age 30 diagnosed with KP based on visual examination that entails observation. The male is administered an topical cream embodiment of the current formulation comprising of about 2.0% DHEA, 5% niacinamide, 1.0% ceramides 1 & 3, 1.0% rosehip oil, 2.0% cholesterol, and 89% phospholipon 90H. The formulation is administered to the male twice a day, namely in the morning and evening. Sufficient cream is applied to the skin so the afflicted area is covered. The user continues with treatment until all physical symptoms of KP (e.g. redness, presence of papules) are no longer visible based on human observation. Samples of the patient's skin are biopsied before treatment begins and then at a monthly basis until the skin is clear. The relative number of sebaceous glands are analyzed using biochemical staining methods such as hematoxylin and eosin staining.

Test of Formulation 2—According to a second exemplary embodiment, a female patient age 35 diagnosed with KP based on visual observation is treated with an embodiment of the current formulation comprising of about 3.0% DHEA, 1.0% ceramides 1 & 3, 1.0% sunflower seed oil, and 2.0% cholesterol and 93% phospholipon 90H. The formulation in gel form is administered to the KP sufferer once a day via topical application, specifically before bedtime. Sufficient gel is applied to the skin so the afflicted area is covered. The user continues with treatment until all physical symptoms of KP (e.g. redness, presence of papules) based on human visual examination is completely diminished. Samples of the patient's skin are biopsied before treatment begins and then at a monthly basis until the skin is clear. Each biopsy sample is evaluated for volume of the sebaceous gland tissue.

Test of Formulation 3—According to a third exemplary embodiment, a female patient age 50 diagnosed with KP based on visual examination is treated with an embodiment of the current formulation comprising of about 4.0% DHEA, 1.0% ceramides 1 & 3, 1.0% squalene and 2.0% cholesterol and 92% phospholipon 90H. The formulation in ointment form is administered to the KP sufferer once a day, specifically before bedtime. Sufficient cream is applied to the skin so the afflicted area is covered. The user continues with treatment until all physical symptoms of KP (e.g. redness, presence of papules) is completely diminished. Samples of the patient's skin are biopsied before treatment begins and then at a monthly basis until the skin is clear. Each biopsy sample is stereologically evaluated for quantification of three dimensional structure of the sebaceous glands.

Test of Formulation 4—According to embodiments of the current invention, a method of producing the formulation is as follows (all percentages are by weight): first, in the water phase, 66% distilled water is combined with 5% niacinaminde powder and 1% vegetable glycerin. The niacinamide powder and glycerin are mixed until dissolved and then heated to 150 degrees Fahrenheit (° F.). Parallel to the water phase, 10% jojoba oil, 2% cranberry seed oil, and 5% emulsifier wax are combined and heated to between 150° F.-170° F. Once both the water phase and oil phase are at or above 150° F., the components of each phase are dissolved and there is less than a 20° F. difference between the two phases, the water and oil phases are combined and mixed together on high sheer for 20 minutes. As the mixture enters the cool down phase (below 110° F.), 3% ceramide cholesterol lipid complex is added. Separately, 3% vegetable glycerin and 2% DHEA are combined until a slightly white slurry is formed. This slurry is added to the mixture containing ceramide cholesterol lipid complex.

### Exemplary Embodiment for Method of Production

| | % by | | |
|---|---|---|---|
| Ingredient | Weight | Phase | Note |
| Distilled Water | 66 | Water Phase | |
| Niacinamide | 5 | Water phase | |
| (powdered; | | | |
| micronized) | | | |
| Vegetable glycerin | 4 | Water phase/ | |
| | | Cool Down | |
| | | phase | |
| Jojoba Oil | 10 | Oil Phase | |
| Cranberry Seed Oil | 2 | Oil Phase | |
| Emulsifier Wax NF | 5 | Oil Phase | |
| Ceramide Cholesterol | 3 | Cool Down | Comprised of 1% ceramides 1 & 3; |
| Complex | | Phase | 2% cholesterol (powedered for |
| | | | cosmetic formulation); 1% sunflower |
| | | | seed oil. Alternatives other than 1:3:1 |
| | | | ratios include 1:2:1; and 1:1:1; 5:1:2. |
| Preservative | 3% | Cool Down | Example includes AMTicide Coconut; |
| | | Phase | phenonip |
| DHEA | 2% | Cool Down | |
| | | Phase | |

According to embodiments, if less DHEA is used by weight (less than 2% by weight), then the oil and water phase components are increased proportionally. Likewise, if more DHEA is used (greater than 2% by weight), the oil and water phase components are decreased proportionally.

One advantage of the current formulation and method is that it obviates the need to treat KP with DHT. DHT is a powerful hormone with potentially harmful side effects. Side effects of DHT include dry skin, changes in your bowel habits, dry mouth, muscle pain, increased thirst, increased urination, high blood pressure, stomach upset, loss of appetite, weight loss, or unusual taste in the mouth. DHT is more potent than DHEA since relatively smaller amounts of DHT are sufficient to stimulate sebaceous gland to produce sebocytes. However, since DHT is relatively potent, there is greater risk of overdosing with a topical application that comprises of DHT.

While multiple embodiments are disclosed, still other embodiments of the present invention will become apparent to those skilled in the art from this detailed description. The invention is capable of myriad modifications in various obvious aspects, all without departing from the spirt and scope of the present invention. Accordingly, the descriptions are to be regarded as illustrative in nature rather than restrictive.

1. A topical cream for the treatment of skin conditions comprising of: dehydroepiandrosterone (DHEA).

2. The topical cream of claim 1, comprising of 0.1%-10% DHEA by weight.

3. The topical cream of claim 1 further comprising at least one additional element from a group comprising of niacinamide, ceramides 1&3, free fatty acids, and cholesterol.

4. The topical cream of claim 3 comprising of 2% DHEA, 5% niacinamide, 1% lipids, 1% cholesterol, and 2% ceramides by weight where the remaining amount is comprised of distilled water.

5. The topical cream of claim 1 comprising of a ceramide cholesterol complex, wherein said complex comprises of ceramides 1 & 3, cholesterol, and a free fatty acid.

6. The topical cream of claim 5 wherein said ceramide cholesterol complex comprises of about 1% ceramides 1 & 3; 2% cholesterol and 1% sunflower seed oil by weight.

7. The topical cream of claim 5 wherein said ceramide cholesterol complex comprises of about 1% ceramides 1 & 3; 3% cholesterol and 1% sunflower seed oil by weight.

8. The topical cream of claim 5 wherein said ceramide cholesterol complex comprises of about 1% ceramides 1 & 3; 1% cholesterol and 1% sunflower seed oil.

9. The topical cream of claim 5 wherein said ceramide cholesterol complex comprises of about 5% ceramides 1 & 3; 1% cholesterol and 2% sunflower seed oil.

10. A topical cream for the treatment of skin conditions comprising of:
DHEA, distilled water, niacinamide, vegetable glycerin, oil, ceramide cholesterol complex, and a preservative.

11. The topical cream of claim 10 further comprising of emulsifier wax NF.

12. The oil of claim 10 wherein such oil is jojoba oil.

13. The oil of claim 10 wherein such oil is cranberry seed oil.

14. The preservative of claim 10 wherein said preservative is AMTicide coconut.

15. The topical cream of claim 10 wherein said cream comprises of 2% DHEA, 5% niacinamide, and at least 3% percent of ceramide cholesterol complex.

16. The topical cream of claim 10 further comprising of alpha-hydroxy acids (AHAs).

17. A method of treating keratosis pilaris wherein the method comprises of:
applying a topical cream between two to three times daily wherein said topical cream comprises of dehydroepiandrosterone (DHEA).

18. The method of claim 16 wherein said topical cream comprises of DHEA and at least one addition from a group comprising of niacinamide, ceramides 1&3, free fatty acids, and cholesterol.

19. The method of treating keratosis pilaris of claim 17 wherein said topical cream is applied after showering.

20. The method of treating keratosis pilaris of claim 17, wherein said method further comprises of pretreatment of the skin with physical or chemical exfoliation.
